(19) United States
(12) Patent Application Publication
(10) Pub. No.: US 2017/0354351 A1
(43) Pub. Date: Dec. 14, 2017

(54) NUTRITION COACHING FOR CHILDREN

(71) Applicant: KONINKLIJKE PHILIPS N.V., EINDHOVEN (NL)

(72) Inventors: JAN MARTIJN KRANS, DEN BOSCH (NL); MARIA HELENA SCHUT, NUENEN (NL); JUDITH HENDRIKA MARIA DE VRIES, BUDEL-SCHOOT (NL); ANASTASIA SCHMALZ, AMSTERDAM (NL)

(73) Assignee: Koninklijke Philips N.V., Eindhoven (NL)

(21) Appl. No.: 15/527,339

(22) PCT Filed: Nov. 20, 2015
(86) PCT No.: PCT/IB2015/059016

§ 371 Date: May 17, 2017

Related U.S. Application Data
(60) Provisional application No. 62/082,850, filed on Nov. 21, 2014.

(51) Int. Cl. A61B 5/11; A61B 5/00; G06F 19/00; G09B 19/00

(52) U.S. Cl. CPC A61B 5/1118; G09B 19/0092; A61B 5/4866; G06F 19/3475; A61B 5/486; A61B 2503/06

(57) ABSTRACT

In an embodiment, an apparatus (42) that provides advice on nutritional and caloric intake requirements for a child based on the child's current growth phase activity behavior and status corresponding to the child's current body mass index, the nutritional requirements determined in terms of a ratio of nutrient components that are tailored to the growth phase of the child.

### FIELD OF THE INVENTION

The present invention is generally related to health management, and more specifically, to nutrition coaching for children based on activity tracking and other recorded data.

### BACKGROUND OF THE INVENTION

A large variety of activity trackers, such as physical activity trackers, is being offered on the market. Such activity trackers may be worn as a bracelet or wristband, and include one or more sensors ranging from a single accelerometer to additional sensors such as heart rate sensors. Typically, the activity tracker is accompanied with an application in a smartphone or other electronics device that provides a dashboard associated with the recorded activity and some data-driven coaching. Some systems utilize the information from the activity trackers to enable feedback on behavioral modification for calorie control, weight control, or general fitness. For instance, U.S. Pat. No. 8,398,546B2 discloses a nutrition and activity management system that monitors energy expenditure of an individual through the use of a body-mounted sensing apparatus. The system also includes a meal planning subsystem that allows a user to customize a meal plan based on individual fitness and weight loss goals. Appropriate foods are recommended to the user based on answers provided to general and medical questionnaires. These questionnaires are used as inputs to the meal plan generation system to ensure that foods are selected that take into consideration specific health conditions or preferences of the user. The system may be provided with functionality to recommend substitution choices based on the food category and exchange values of the food and will match the caloric content between substitutions. The system may be further adapted to generate a list of food or diet supplement intake recommendations based on answers provided by the user to a questionnaire.

### SUMMARY OF THE INVENTION

One object of the present invention is provide nutritional advice that contemplates various growth phases of a child as well as activity behavior and nutritional requirements within the current growth phase. To better address such concerns, in a first aspect of the invention, an apparatus is presented that provides advice on nutritional and caloric intake requirements for a child based on the child's current growth phase activity behavior and status corresponding to the child's current body mass index, the nutritional requirements determined in terms of a ratio of nutrient components that are tailored to the growth phase of the child.

In an embodiment, a processing circuit of the apparatus is configured to provide meal planning recommendations personalized for the child based on the nutritional requirements and the caloric intake requirements, the meal planning recommendations comprising one or any combination of the following: food selection, food preparation, meal timing, food ingredients, food portions, relative food proportion, nutrient levels, and proportion of nutrients. The tailoring of the meal plans recognizes that, though adults and children have nutritional needs that are similar in principle (e.g., both groups need the same types of nutrients, such as vitamins, minerals, carbohydrates, protein, fat), children require a different amount of specific nutrients at different phases of growth, and the ratio between different nutrients changes over the various growth or development phases and among genders. In other words, the meal plans that are recommended are distinct from meal plan systems for adults.

In an embodiment, the processing circuit is further configured to provide the meal planning recommendations based on additional input, wherein the meal planning recommendations for the child in a first growth phase of the plurality of growth phases are different than the meal planning recommendations for the child in a second growth phase of the plurality of growth phases. Aside from recognizing the differences in nutrient requirements and ratios of nutrients among different growth phases, there is also a recognition that children also differ in terms of the likes and dislikes and that food allergies are more prominent in children, where having input in the form of questionnaires via web page or other input (e.g., phone survey, email, etc.) and/or export/import from other databases enables a determination of appropriate meal plans and suitable substitutes consistent with the nutritional requirements and caloric intake.

In an embodiment, the costs are pre-defined, estimated from responses from the subject, based on a questionnaire or interview of the subject, or based on any combination of the predefinition, responses, questionnaire and interview. Recognizing the value in establishing a cost by one or a combination of various mechanisms enables a realization of a cost component in deriving a health plan as opposed to an inefficient trial and error approach to finding a time most suitable for adding the physical activity.

In an embodiment, a processing circuit of the apparatus is configured to determine additional health plans as well as the health plan as options for selection, the additional health plans corresponding to the series of forecasted measurements of the physiological parameter that minimizes the total cost for the physical activity while maximizing the health benefit associated with the physiological parameter. The presentation of additional options allows the subject more options in choosing among optimized plans, as opposed to a more generalized approach to health plans.

In an embodiment, the processing circuit is further configured to determine the nutritional requirements and caloric intake requirements based on computing and comparing growth rates of the child over plural periods of time. For instance, further differences among children of the same age include the timing of growth spurts, as evidenced by the exceptions noted in middle school, for instance, of boys that can grow a mustache or stand above others for at least until later high school years. The processing circuit can compute a current growth data and the growth rate for one or more periods in the past and compare the current rate of growth with past growth rates to ascertain whether the child is currently in a growth spurt.

In an embodiment, the growth data includes one or any combination of weight, height, body mass index, gender, age, and girth of the child. For instance, by including the current body mass index (whether received or derived from the other growth data), the processing circuit can tailor the nutrients and caloric needs to a weight goal of the child.

In an embodiment, the processing circuit is configured to receive activity behavior data based on manual input, sensor data, or a combination of manual input and sensor data, wherein the processing circuit determines that the activity behavior falls within one of plural predefined categories of activity levels based on the activity behavior data. For instance, the processing circuit makes a determination as to whether the behavior data suggests that the child is sedentary, normally active, very active, etc., enabling the tailoring of caloric needs to historical activity levels of the child.

In an embodiment, the processing circuit is further configured to cause an automated ordering of food corresponding to the meal planning recommendations. For instance, the processing circuit may automatically (or based on a grant of permission solicited in a web-prompt or other mechanism of communication) generate a grocery list for receipt by a meal delivery service or grocer, providing a mechanism to further bolster compliance with the plan without the temptation to stray from the plan that shopping for food in person (or worse, with the child) may cause.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram that illustrates an example nutrition coaching system in accordance with an embodiment of the invention.

FIG. 2 is a schematic diagram that illustrates an example environment in which a nutrition coaching system is used in accordance with an embodiment of the invention.

FIG. 3 is a block diagram that illustrates circuitry for an example wearable device in accordance with an embodiment of the invention.

FIG. 4 is a block diagram that illustrates a processing circuit for an example computing device in accordance with an embodiment of the invention.

FIGS. 5A-5B are schematic diagrams that graphically illustrate an example process by which the nutrition coaching system receives and provides personalized advice on nutrient and caloric needs in accordance with an embodiment of the invention.

FIGS. 5C-5D are schematic diagrams of stature for age and weight for age percentiles published by the Center for Disease Control for boys and girls.

FIG. 6 is a flow diagram that illustrates a nutrition coaching method in accordance with an embodiment of the invention.

### DETAILED DESCRIPTION OF EMBODIMENTS

Disclosed herein are certain embodiments of a nutrition coaching system and method that provides a nutrition advice service for a child that is personalized based at least in part on an analysis of the child's lifestyle and physical development. Activity behavior of the child and other child data, such as height, weight, age, gender, body mass index (BMI) are recorded, and used to make various determinations leading up to the personalized nutrition device. For instance, based on the recorded data, which may be received (directly or indirectly) from sensors of a wearable device worn by the child and/or other devices or systems or manually input (e.g., from a parent and/or the child depending on the ability of the child), the growth phase of the child, the relative weight status (e.g., overweight, underweight, normal), and activity level are all determined and used to tailor the nutritional needs to the child, enabling the provision of personalized advice for the child.

Digressing briefly, although some conventional systems integrate the wearable device technology with the provision of nutritional advice, such systems are focused primarily on adults, with insufficient accommodations for handling the particular needs of children. For instance, children go through distinct periods or phases of development corresponding to babies, toddlers, preschoolers, school age children, and teenagers. During each of these phases, multiple changes in the development of the brain of the child are taking place, with the timing of such changes and the scope of the changes genetically determined. Accordingly, parenting challenges and needs differ per phase of child development. Adding to the complexity of nutritional advice for children based on differences in brain development is the fact that children also develop differently physically, such as by growing in height, growing in weight, growing in shoe size, etc. And, the growth is not linear over time, but rather, more step-like (e.g., according to growth spurts). Average child-growth graphs exist (height, weight vs time, such as those developed by the National Center for Health Statistics in collaboration with the National Center for Chronic Disease Prevention and Health Promotion, accessed via the CDC web for growth rates), from which general guidelines on child weight gain can be obtained. But large differences exist in growth rates and growth periods between children (e.g., based on DNA, lifestyle, growth spurt, etc.). Further, some online calories requirement calculators (aka, healthy calculators with calories intake requirements) are available which take into account height, weight, and age, as well as advice on calories that are dependent on activity level of the person and nutritional recommendations in terms of nutritional components (e.g., carbohydrates, proteins, fats), yet the ratio between these nutritional components is taken as constant over all age groups. Further, some charts, such as those available via the 2010 U.S. Dietary Guidelines for Americans (appendices 5 and 6) provide for proper ratios of nutrients, yet lump children in several age groups that is not unlike assuming that because a shirt has a tag attached that says for 1-3 year olds, that the short should fit all 1-3 year olds. Children differ in the rate of growth even within a given age group. In contrast to the existing charts and/or systems, certain embodiments of nutrition coaching systems monitor the activity behavior of the child as well as the growth phases of the child using wearable devices and other apparatuses and/or systems, enabling a tailoring of nutrient and caloric intake requirements specific to the needs of the child, and corresponding advice.

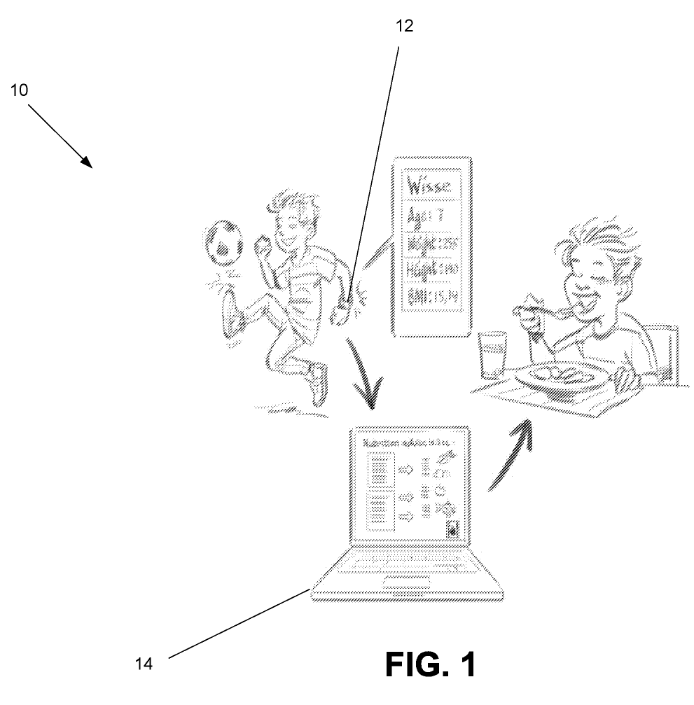

Having provided a general summary of certain embodiments of a nutrition coaching system, attention is directed to FIG. 1, which illustrates an example nutrition coaching system 10 in accordance with an embodiment of the invention. The nutrition coaching system 10 comprises a wearable device 12 and a computing device 14, though in some embodiments, the nutrition coaching system 10 may comprise additional or fewer components. As shown, a child typically spends the day according to various levels of activity behavior, and as shown in FIG. 1, is engaged in playing soccer and at another point in the day, is eating. The child wears the wearable device 12, which monitors and/or tracks the activity of the child (e.g., fitness, activity, sleep, eating activity, etc.), as well as various parameters, which may include heart rate, steps, motion of limbs, respiration, etc. The nutrition coaching system 10 may also include a growth tracking device (e.g., tracking one or more dimensions of the child, such as weight, girth, height, etc., such as a weight scale), or information from such a growth tracking device. The growth tracking device may be used to provide growth data associated with the child, such as weight and/or height. The nutrition coaching system 10 may also receive input from other mechanisms, such as via the parent, the child, or via child records, which may also provide the same and/or other growth data, such as age and gender. That is, one or more of the information corresponding to the growth data may be communicated to the nutrition coaching system 10 via the computing device 14, such as via manual input by the child, a parent, or other relative or guardian, or shared by one or more institutions (e.g., a medical facility, medical records facility, etc.), and/or in some embodiments, recorded (e.g., body mass index, or parameters used to determine body mass index) from the wearable device 12 or a growth tracking device. In an embodiment, the nutrition coaching system 10 may provide nutrition advice to the child (or a parent, guardian, etc.) based on an analysis of the inputted and/or recorded data and determine the nutritional needs of the child in a particular growth phase in life. By analysis of the data, personalized advice (recommendations) may be given by the nutrition coaching system 10 regarding a well-balanced nutrition and required caloric intake for the child. Such advice may be used as guidance for meal planning, such as selection, preparation, timing, and portioning.

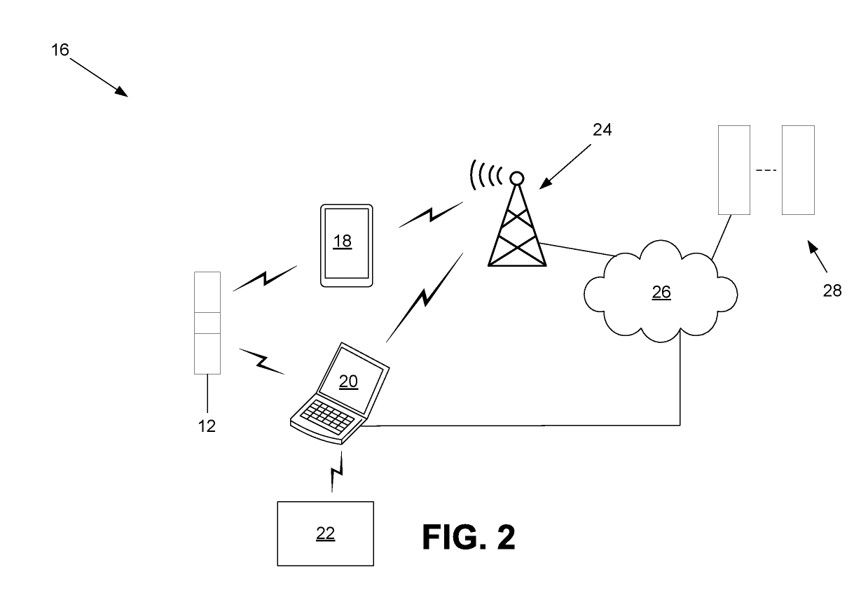

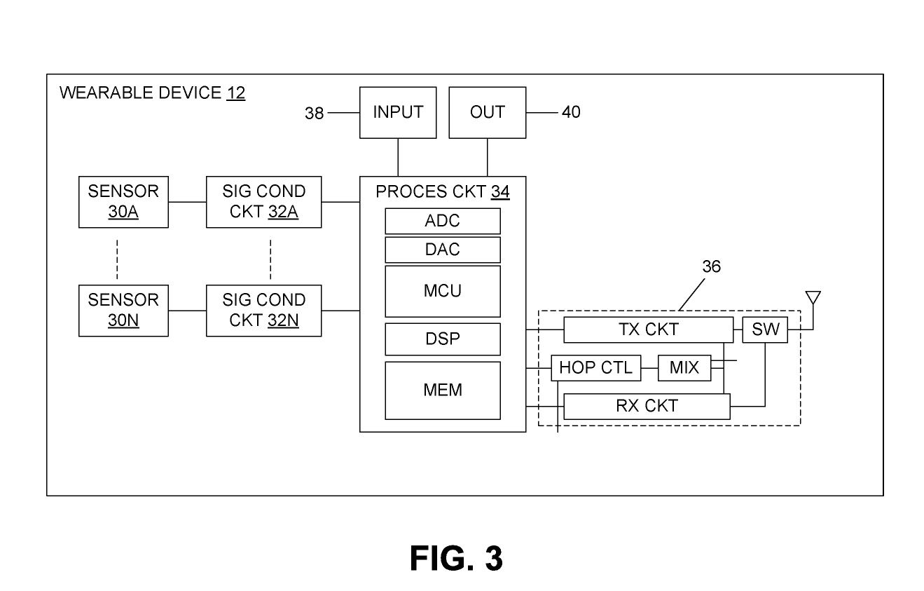

FIGS. 2-4 provide an example system environment and associated components of that environment to facilitate operations of an embodiment of a nutrition coaching system, similar to the nutrition coaching system 10 of FIG. 1. Referring to FIG. 2, shown is an example environment 16 in which a nutrition coaching system may be used. It should be appreciated by one having ordinary skill in the art in the context of the present disclosure that the environment 16 is one example among many, and that some embodiments of a health coaching system may be used in environments with fewer, greater, and/or different components that those depicted in FIG. 2. The environment 16 comprises a plurality of devices that enable communication of information throughout one or more networks. The depicted environment 16 comprises the wearable device 12, electronics devices 18, 20, a growth tracking device 22 (which may be an electronics device or mechanical device in some embodiments), a cellular network 24, a wide area network 26 (e.g., also described herein as the Internet), and a remote computing system 28. The wearable device 12, as described further in association with FIG. 3, is typically worn by the child (e.g., around the wrist), and comprises a plurality of sensors that track physical activity (e.g., activity behavior) of the child (e.g., steps, swim strokes, pedaling strokes, etc.), sense or derive physiological parameters (e.g., heart rate, respiration, skin temperature, etc.) based on the sensor data, and optionally sense various other parameters (e.g., outdoor temperature, humidity, location, etc.) pertaining to the surrounding environment of the wearable device 12. A representation of such gathered data may be communicated to the child via an integrated display on the wearable device 12 and/or on another device or devices.

Also, such data gathered by the wearable device 12 may be communicated (e.g., continually, periodically, and/or aperiodically) to one or more electronics devices, such as the electronics devices 18 and 20. Such communication may be achieved wirelessly (e.g., using near field communications (NFC) functionality, Blue-tooth functionality, etc.) and/or according to a wired medium (e.g., universal serial bus (USB), etc.). In the depicted example, the electronics device 18 is embodied as a phone and the electronics device 20 is embodied as a computer. It will be assumed that the growth tracking device 22 (e.g., a weigh scale, though in some embodiments, may monitor/track other growth data, such as girth, body mass index, height, etc.) is an electronics device with communications capability and an architecture (e.g., a processor and memory) somewhat similar to the electronics devices 18 and/or 20, though it should be appreciated by one having ordinary skill in the art in the context of the present disclosure that any information obtained from the growth tracking device 22 may be communicated to the electronics device 20 (and/or electronics device 18) via manual input. It should be appreciated that although each electronics device is listed in the singular, some implementations may utilize different quantities for each of the electronics devices 18, 20. Further, in some embodiments, fewer, additional, and/or other types of electronics devices may be used. The phone 18 may be embodied as a smartphone, mobile phone, cellular phone, pager, among other handheld computing/communication devices with telephony functionality. For the sake of example, assume the phone 18 is embodied as a smartphone. The smartphone 18 comprises at least two different processors, including a baseband processor and an application processor. The baseband processor comprises a dedicated processor for deploying functionality associated with a protocol stack, such as a GSM (Global System for Mobile communications) protocol stack. The application processor comprises a multi-core processor for providing a user interface and running applications. The baseband processor and application processor have respective associated memory (e.g., random access memory (RAM), Flash memory, etc.), peripherals, and a running clock.

More particularly, the baseband processor may deploy functionality of a GSM protocol stack to enable the smartphone 18 to access one or a plurality of wireless network technologies, including WCDMA (Wideband Code Division Multiple Access), CDMA (Code Division Multiple Access), EDGE (Enhanced Data Rates for GSM Evolution), GPRS (General Packet Radio Service), Zigbee (e.g., based on IEEE 802.15.4), Bluetooth, Wi-Fi (Wireless Fidelity, such as based on IEEE 802.11), and/or LTE (Long Term Evolution), among variations thereof and/or other telecommunication protocols, standards, and/or specifications. The baseband processor manages radio communications and control functions, including signal modulation, radio frequency shifting, and encoding. The baseband processor may comprise a GSM modem having one or more antennas, a radio (e.g., RF front end), and analog and digital baseband circuitry. The RF front end comprises a transceiver and a power amplifier to enable the receiving and transmitting of signals of a plurality of different frequencies, enabling access to the cellular network 24. The analog baseband is coupled to the radio and provides an interface between the analog and digital domains of the GSM modem. The analog baseband comprises circuitry including an analog-to-digital converter (ADC) and digital-to-analog converter (DAC), as well as control and power management/distribution components and an audio codec to process analog and/or digital signals received from the smartphone user interface (e.g., microphone, earpiece, ring tone, vibrator circuits, etc.). The ADC digitizes any analog signals for processing by the digital baseband processor. The digital baseband processor deploys the functionality of one or more levels of the GSM protocol stack (e.g., Layer 1, Layer 2, etc.), and comprises a microcontroller (e.g., microcontroller unit or MCU) and a digital signal processor (DSP) that communicate over a shared memory interface (the memory comprising data and control information and parameters that instruct the actions to be taken on the data processed by the application processor). The MCU may be embodied as a RISC (reduced instruction set computer) machine that runs a real-time operating system (RTIOS), with cores having a plurality of peripherals (e.g., circuitry packaged as integrated circuits) such as RTC (real-time clock), SPI (serial peripheral interface), I2C (inter-integrated circuit), UARTs (Universal Asynchronous Receiver/Transmitter), devices based on IrDA (Infrared Data Association), SD/MMC (Secure Digital/Multimedia Cards) card controller, keypad scan controller, and USB devices, GPRS crypto module, TDMA (Time Division Multiple Access), smart card reader interface (e.g., for the one or more SIM (Subscriber Identity Module) cards), timers, and among others. For receive-side functionality, the MCU instructs the DSP to receive, for instance, in-phase/quadrature (I/Q) samples from the analog baseband and perform detection, demodulation, and decoding with reporting back to the MCU. For transmit-side functionality, the MCU presents transmittable data and auxiliary information to the DSP, which encodes the data and provides to the analog baseband (e.g., converted to analog signals by the DAC). The application processor may be embodied as a System on a Chip (SOC), and supports a plurality of multimedia related features including web browsing to access one or more computing devices of the computing system 28 that are coupled to the Internet, email, multimedia entertainment, games, etc.

The application processor includes an operating system that enables the implementation of a plurality of user applications. For instance, the application processor may deploy interface software (e.g., middleware, such as a browser with or operable in association with one or more application program interfaces (APIs)) to enable access to a cloud computing framework or other networks to provide remote data access/storage/processing, and through cooperation with an embedded operating system, access to calendars, location services, reminders, etc. For instance, in some embodiments, the nutrition coaching system may operate using cloud computing, where the processing and storage of growth data, activity behavior data, and nutrition data and the determination of nutritional requirements and caloric intake requirements and provision of advice may be achieved by one or more devices of the computing system 28. The application processor generally comprises a processor core (Advanced RISC Machine or ARM), multimedia modules (for decoding/encoding pictures, video, and/or audio), a graphics processing unit (GPU), wireless interfaces, and device interfaces. The wireless interfaces may include a Bluetooth or Zigbee module(s) that enables wireless communication with the wearable device 12 or other local devices, a Wi-Fi module for interfacing with a local 802.11 network, and a GSM module for access to the cellular network 24 and the wide area network 26. The device interfaces coupled to the application processor may include a respective interface for such devices as a display screen. The display screen may be embodied in one of several available technologies, including LCD or Liquid Crystal Display (or variants thereof, such as Thin Film Transistor (TFT) LCD, In Plane Switching (IPS) LCD)), light-emitting diode (LED)-based technology, such as organic LED (OLED), Active-Matrix OLED (AMOLED), or retina or haptic-based technology. For instance, the display screen may be used to present web pages and/or other documents received from the computing system 28 and/or in some embodiments (e.g., for local processing) graphic user interfaces (GUIs) rendered locally, either of which may present feedback in the form of a visual representation of the nutritional and caloric intake advice and/or meal plans, as described further below. Other interfaces include a keypad, USB (Universal Serial Bus), SD/MMC card, camera, GPRS, Wi-Fi, GPS, and/or FM radios, memory, among other devices. It should be appreciated by one having ordinary skill in the art, in the context of the present disclosure, that variations to the above may be deployed in some embodiments to achieve similar functionality.

The computer 20 may be embodied as a laptop, personal computer, workstation, personal digital assistant, tablet, among other computing devices with communication capability. The computer 20 may be in wireless or wired (e.g., temporarily, such as via USB connection, or persistently, such as an Internet connection or local area network connection) communication with other devices (e.g., the phone 18, the growth tracking device 22, etc.). The computer 20 may include similar hardware and software/firmware to that described above for the phone 18 to enable access to wireless and/or cellular networks (e.g., through communication cards comprising radio and/or cellular modem functionality) and/or other devices (e.g., Bluetooth transceivers, NFC transceivers, etc.), such as wireless or (temporary) wired connection to the wearable device 12. In some implementations, the computer 20 may be coupled to the Internet 26 through the plain old telephone service (POTS), using technologies such as digital subscriber line (DSL), asymmetric DSL (ADSL), and/or according to broadband technology that uses a coaxial, twisted pair, and/or fiber optic medium. Discussion of such communication functionality is omitted here for brevity. Generally, in terms of hardware architecture, the computer 20 includes a processor, memory, and one or more input and/or output (I/O) devices (or peripherals) that are communicatively coupled via a local interface. The local interface can be, for example but not limited to, one or more buses or other wired or wireless connections. The local interface may have additional elements, which are omitted for brevity, such as controllers, buffers (caches), drivers, repeaters, and receivers, to enable communications. Further, the local interface may include address, control, and/or data connections to enable appropriate communications among the aforementioned components.

The processor is a hardware device for executing software, particularly that stored in memory. The processor can be any custom made or commercially available processor, a central processing unit (CPU), an auxiliary processor among several processors associated with the computing device 14, a semiconductor based microprocessor (in the form of a microchip or chip set), a macroprocessor, or generally any device for executing software instructions.

The memory can include any one or combination of volatile memory elements (e.g., random access memory (RAM, such as DRAM, SRAM, SDRAM, etc.) and nonvolatile memory elements (e.g., ROM, hard drive, Flash, EPROM, EEPROM, CDROM, etc.). Moreover, the memory may incorporate electronic, magnetic, optical, semi-conductive, and/or other types of storage media. Note that the memory can have a distributed architecture, where various components are situated remote from one another, but can be accessed by the processor.

The software in memory may include one or more separate programs, such as interface software (e.g., middleware, such as browser software with or associated with one or more APIs) to communicate with other network devices, such as one or more devices of the computing system 28, the separate programs each comprising an ordered listing of executable instructions for implementing logical functions. The software in the memory also includes application software and a suitable operating system (O/S). The operating system may be embodied as a Windows operating system available from Microsoft Corporation, a Macintosh operating system available from Apple Computer, a UNIX operating system, among others. The operating system essentially controls the execution of other computer programs, and provides scheduling, input-output control, file and data management, memory management, and communication control and related services.

The I/O devices may include input devices, for example but not limited to, a keyboard, mouse, scanner, microphone, etc. Furthermore, the I/O devices may also include output devices, for example but not limited to, a printer, display, etc. For instance, the I/O devices embodied as a display screen may be used to present web pages and/or other documents received from the computing system 28 and/or in some embodiments (e.g., for local processing) graphic user interfaces (GUIs) rendered locally, either of which may present feedback in the form of a visual representation of the nutritional and caloric intake advice and/or meal plans, as described further below. The display screen may be configured according to any one of a variety of technologies, including cathode ray tube (CRT), liquid crystal display (LCD), plasma, haptic, among others well-known to those having ordinary skill in the art.

If the computer is a PC, workstation, or the like, the software in the memory may further include a basic input output system (BIOS). The BIOS is a set of essential software routines that initialize and test hardware at startup, start the O/S, and support the transfer of data among the hardware devices. The BIOS is stored in ROM so that the BIOS can be executed when the computer 20 is activated.

When the computer 20 is in operation, the processor is configured to execute the software stored within the memory, to communicate data to and from the memory, and to generally control operations of the computer 20 pursuant to the software. Software can be stored on any non-transitory computer readable medium for use by or in connection with any computer related system or method. In the context of this document, a computer readable medium comprises an electronic, magnetic, optical, electromagnetic, infrared, or semiconductor system, apparatus, device or means that can contain or store a computer program for use by or in connection with a computer related system or method. The software can be embodied in any non-transitory computer-readable medium for use by or in connection with an instruction execution system, apparatus, or device, such as a computer-based system, processor-containing system, or other system that can fetch the instructions from the instruction execution system, apparatus, or device and execute the instructions.

The cellular network 24 may include the necessary infrastructure to enable cellular communications by the phone 18 and optionally the computer 20 (and in some embodiments, the growth tracking device 22). There are a number of different digital cellular technologies suitable for use in the cellular network 24, including: GSM, GPRS, CDMAOne, CDMA2000, Evolution-Data Optimized (EV-DO), EDGE, Universal Mobile Telecommunications System (UMTS), Digital Enhanced Cordless Telecommunications (DECT), Digital AMPS (IS-136/TDMA), and Integrated Digital Enhanced Network (iDEN), among others.

The wide area network 26 may comprise one or a plurality of networks that in whole or in part comprise the Internet. The electronics devices 18, 20 access the devices of the computing system 28 via the Internet 26, which may be further enabled through access to one or more networks including PSTN (Public Switched Telephone Networks POTS, Integrated Services Digital Network (ISDN), Ethernet, Fiber, DSL/ADSL, among others.

The computing system 28 comprises a plurality of devices coupled to the wide area network 26, including one or more computing devices such as application servers, a computer network, and data storage. As described previously, the computing system 28 may serve as a cloud computing environment (or other server network) for the electronics devices 18, 20, performing processing and data storage on behalf of (or in some embodiments, in addition to) the electronics devices 18, 20. In some embodiments, one or more of the functionality of the computing system 28 may be performed at the respective electronics devices 18, 20.

An embodiment of a nutrition coaching system may comprise one or more devices (or equivalently, one or more apparatuses) of the computing system 28, or in some embodiments, a combination of one or more of the electronics devices 18, 20, 22 and one or more devices of the computing system 28 or in some embodiments, a combination of the wearable device 12, one or more of the electronics devices 18, 20, 22, and one or more devices of the computing system 28. In some embodiments, the nutrition coaching system functionality may be carried out locally, such as via one or more of (e.g., one of either of the devices 18, 20, 22, or a combination of two or more of) the electronics devices 18, 20, 22, or a combination of the one or more of the electronics devices 18, 20, 22 and the wearable device 12.

Having generally described an example environment 16 in which an embodiment of a nutrition coaching system may be implemented, attention is directed to FIG. 3. FIG. 3 illustrates example circuitry for the example wearable device 12, and in particular, underlying circuitry and software (e.g., architecture) of the wearable device 12. It should be appreciated by one having ordinary skill in the art in the context of the present disclosure that the architecture of the wearable device 12 depicted in FIG. 3 is but one example, and that in some embodiments, additional, fewer, and/or different components may be used to achieve similar and/or additional functionality. In one embodiment, the wearable device 12 comprises a plurality of sensors 30 (e.g., 30A-30N), one or more signal conditioning circuits 32 (e.g., SIG COND CKT 32A-SIG COND CKT 32N) coupled respectively to the sensors 30, and a processing circuit 34 (PROCES CKT) that receives the conditioned signals from the signal conditioning circuits 32. In one embodiment, the processing circuit 34 comprises an analog-to-digital converter (ADC), a digital-to-analog converter (DAC), a microcontroller (e.g., MCU), a digital signal processor (DSP), and memory (MEM). In some embodiments, the processing circuit 34 may comprise fewer or additional components than those depicted in FIG. 3. For instance, in one embodiment, the processing circuit 34 may consist of the microcontroller. The memory comprises an operating system (OS) and application software. The application software comprises a plurality of algorithms (e.g., application modules of executable code) to process the signals (and associated data) measured by the sensors and record and/or derive physiological parameters, such as heart rate, blood pressure, respiration, perspiration, etc. The application software also comprises communications software, such as that used to enable the wearable device 12 to operate according to one or more of a plurality of different communication technologies (e.g., NFC, Bluetooth, Wi-Fi, Zigbee, etc.). In some embodiments, the communications software may be in separate or other memory.

The memory further comprises one or more data structures. In one embodiment, the processing circuit 34 is coupled to a communications circuit 36. The communications circuit 36 serves to enable wireless communications between the wearable device 12 and other electronics devices, such as the phone 18, the laptop 20, and/or other devices. The communications circuit 36 is depicted as a Bluetooth circuit, though not limited to this transceiver configuration. For instance, in some embodiments, the communications circuit 36 may be embodied as any one or a combination of an NFC circuit, Wi-Fi circuit, transceiver circuitry based on Zigbee, among others such as optical or ultrasonic based technologies. The processing circuit 34 is further coupled to input/output (I/O) devices or peripherals, such as an input interface 38 (INPUT) and output interface 40 (OUT). Note that in some embodiments, functionality for one or more of the aforementioned circuits and/or software may be combined into fewer components/modules, or in some embodiments, further distributed among additional components/modules. For instance, the processing circuit 34 may be packaged as an integrated circuit that includes the microcontroller, the DSP, and memory, whereas the ADC and DAC may be packaged as a separate integrated circuit coupled to the processing circuit 34. In some embodiments, one or more of the functionality for the above-listed components may be combined, such as functionality of the DSP performed by the microcontroller.

The sensors 30 are selected to perform detection and measurement of a plurality of physiological and activity behavioral parameters, including heart rate, heart rate variability, heart rate recovery, blood flow rate, activity level, muscle activity (e.g., movement of limbs, repetitive movement, core movement, body orientation/position, power, speed, acceleration, etc.), muscle tension, blood volume, blood pressure, blood oxygen saturation, respiratory rate, perspiration, skin temperature, body weight, and body composition (e.g., body mass index or BMI). The sensors 30 may be embodied as inertial sensors (e.g., gyroscopes, single or multi-axis accelerometers, such as those using piezoelectric, piezoresistive or capacitive technology in a microelectromechanical system (MEMS) infrastructure), flex and/or force sensors (e.g., using variable resistance), electromyographic sensors, electrocardiographic sensors (e.g., EKG, ECG) magnetic sensors, photoplethysmographic (PPG) sensors, bio-impedance sensors, infrared proximity sensors, acoustic/ultrasonic/audio sensors, a strain gauge, galvanic skin/sweat sensors, pH sensors, temperature sensors, pressure sensors, and photocells. In some embodiments, other types of sensors 30 may be used to facilitate health and/or fitness related computations, including a global navigation satellite systems (GNSS) sensor (e.g., global positioning system (GPS) receiver) to facilitate determinations of distance, speed, acceleration, location, altitude, etc. (e.g., location data and movement), barometric pressure, humidity, outdoor temperature, etc. In some embodiments, GNSS functionality may be achieved via the communications circuit 36 or other circuits coupled to the processing circuit 34.

The signal conditioning circuits 32 include amplifiers and filters, among other signal conditioning components, to condition the sensed signals including data corresponding to the sensed physiological parameters before further processing is implemented at the processing circuit 34. Though depicted in FIG. 3 as respectively associated with each sensor 30, in some embodiments, fewer signal conditioning circuits 32 may be used (e.g., shared for more than one sensor 30). In some embodiments, the signal conditioning circuits 32 (or functionality thereof) may be incorporated elsewhere, such as in the circuitry of the respective sensors 30 or in the processing circuit 34 (or in components residing therein). Further, although described above as involving unidirectional signal flow (e.g., from the sensor 30 to the signal conditioning circuit 32), in some embodiments, signal flow may be bi-directional. For instance, in the case of optical measurements, the microcontroller may cause an optical signal to be emitted from a light source (e.g., light emitting diode(s) or LED(s)) in or coupled to the circuitry of the sensor 30, with the sensor 30 (e.g., photocell) receiving the reflected/refracted signals.

The communications circuit 36 is managed and controlled by the processing circuit 34. The communications circuit 36 is used to wirelessly interface with the electronics devices 18, 20 (FIG. 2). In one embodiment, the communications circuit 36 may be configured as a Bluetooth transceiver, though in some embodiments, other and/or additional technologies may be used, such as Wi-Fi, Zigbee, NFC, among others. In the embodiment depicted in FIG. 3, the communications circuit 36 comprises a transmitter circuit (TX CKT), a switch (SW), an antenna, a receiver circuit (RX CKT), a mixing circuit (MIX), and a frequency hopping controller (HOP CTL). The transmitter circuit and the receiver circuit comprise components suitable for providing respective transmission and reception of an RF signal, including a modulator/demodulator, filters, and amplifiers. In some embodiments, demodulation/modulation and/or filtering may be performed in part or in whole by the DSP. The switch switches between receiving and transmitting modes. The mixing circuit may be embodied as a frequency synthesizer and frequency mixers, as controlled by the processing circuit 34. The frequency hopping controller controls the hopping frequency of a transmitted signal based on feedback from a modulator of the transmitter circuit. In some embodiments, functionality for the frequency hopping controller may be implemented by the microcontroller or DSP. Control for the communications circuit 36 may be implemented by the microcontroller, the DSP, or a combination of both. In some embodiments, the communications circuit 36 may have its own dedicated controller that is supervised and/or managed by the microcontroller.

In operation, a signal (e.g., at 2.4 GHz) may be received at the antenna and directed by the switch to the receiver circuit. The receiver circuit, in cooperation with the mixing circuit, converts the received signal into an intermediate frequency (IF) signal under frequency hopping control attributed by the frequency hopping controller and then to baseband for further processing by the ADC. On the transmitting side, the baseband signal (e.g., from the DAC of the processing circuit 34) is converted to an IF signal and then RF by the transmitter circuit operating in cooperation with the mixing circuit, with the RF signal passed through the switch and emitted from the antenna under frequency hopping control provided by the frequency hopping controller. The modulator and demodulator of the transmitter and receiver circuits may be frequency shift keying (FSK) type modulation/demodulation, though not limited to this type of modulation/demodulation, which enables the conversion between IF and baseband. In some embodiments, demodulation/modulation and/or filtering may be performed in part or in whole by the DSP. The memory stores firmware that is executed by the microcontroller to control the Bluetooth transmission/reception.

Though the communications circuit 36 is depicted as an IF-type transceiver, in some embodiments, a direct conversion architecture may be implemented. As noted above, the communications circuit 36 may be embodied according to other and/or additional transceiver technologies, such as NFC, Wi-Fi, or Zigbee.

The processing circuit 34 is depicted in FIG. 3 as including the ADC and DAC. For sensing functionality, the ADC converts the conditioned signal from the signal conditioning circuit 32 and digitizes the signal for further processing by the microcontroller and/or DSP. The ADC may also be used to convert analogs inputs that are received via the input interface 38 to a digital format for further processing by the microcontroller. The ADC may also be used in baseband processing of signals received via the communications circuit 36. The DAC converts digital information to analog information. Its role for sensing functionality may be to control the emission of signals, such as optical signals or acoustic signal, from the sensors 30. The DAC may further be used to cause the output of analog signals from the output interface 40. Also, the DAC may be used to convert the digital information and/or instructions from the microcontroller and/or DSP to analog signal that are fed to the transmitter circuit. In some embodiments, additional conversion circuits may be used.

The microcontroller and the DSP provide the processing functionality for the wearable device 12. In some embodiments, functionality of both processors may be combined into a single processor, or further distributed among additional processors. The DSP provides for specialized digital signal processing, and enables an offloading of processing load from the microcontroller. The DSP may be embodied in specialized integrated circuit(s) or as field programmable gate arrays (FPGAs). In one embodiment, the DSP comprises a pipelined architecture, with comprises a central processing unit (CPU), plural circular buffers and separate program and data memories according to a Harvard architecture. The DSP further comprises dual busses, enabling concurrent instruction and data fetches. The DSP may also comprise an instruction cache and I/O controller, such as those found in Analog Devices SHARC® DSPs, though other manufacturers of DSPs may be used (e.g., Freescale multi-core MSC81xx family, Texas Instruments C6000 series, etc.). The DSP is generally utilized for math manipulations using registers and math components that may include a multiplier, arithmetic logic unit (ALU, which performs addition, subtraction, absolute value, logical operations, conversion between fixed and floating point units, etc.), and a barrel shifter. The ability of the DSP to implement fast multiply-accumulates (MACs) enables efficient execution of Fast Fourier Transforms (FFTs) and Finite Impulse Response (FIR) filtering. The DSP generally serves an encoding and decoding function in the wearable device 12. For instance, encoding functionality may involve encoding commands or data corresponding to transfer of information to the electronics devices 18, 20. Also, decoding functionality may involve decoding the information received from the sensors 30 (e.g., after processing by the ADC).

The microcontroller comprises a hardware device for executing software/firmware, particularly that stored in memory. The microcontroller can be any custom made or commercially available processor, a central processing unit (CPU), a semiconductor based microprocessor (in the form of a microchip or chip set), a macroprocessor, or generally any device for executing software instructions. Examples of suitable commercially available microprocessors include Intel's® Itanium® and Atom® microprocessors, to name a few non-limiting examples. The microcontroller provides for management and control of the wearable device 12, including determining physiological parameters based on the sensors 30, and for enabling communication with the electronics devices 18, 20.

The memory can include any one or combination of volatile memory elements (e.g., random access memory (RAM, such as DRAM, SRAM, SDRAM, etc.)) and nonvolatile memory elements (e.g., ROM, Flash, solid state, EPROM, EEPROM, etc.). Moreover, the memory may incorporate electronic, magnetic, and/or other types of storage media.

The software in memory may include one or more separate programs, each of which comprises an ordered listing of executable instructions for implementing logical functions. In the example of FIG. 3, the software in the memory includes a suitable operating system and application software that includes a plurality of algorithms for determining physiological and/or activity behavioral measures and/or other information or data (e.g., such as location) based on the output from the sensors 30. The raw data from the sensors 30 may be used by the algorithms to determine various physiological and/or behavioral measures (e.g., heart rate, biomechanics, such as swinging of the arms), and may also be used to derive other parameters, such as energy expenditure, heart rate recovery, aerobic capacity (e.g., VO2 max, etc.), among other derived measures of physical performance. In some embodiments, these derived parameters may be computed externally (e.g., at the electronics devices 18, 20 or one or more devices of the computing system 28) in lieu of, or in addition to, the computations performed local to the wearable device 12. The application software may also include communications software to enable communications with other electronics devices. The operating system essentially controls the execution of other computer programs, such as the application software and communications software, and provides scheduling, input-output control, file and data management, memory management, and communication control and related services. The memory may also include a data structure, which includes user data, such as weight, height, age, gender, body mass index (BMI) that is used by the microcontroller executing the executable code of the algorithm to accurately interpret the measured physiological and/or behavioral data. In some embodiments, the data structure of user data may be stored elsewhere, such as at the electronics devices 18, 20 and/or at one or more devices of the computing system 28 in lieu of, or in addition to being stored at the wearable device 12.

The software in memory comprises a source program, executable program (object code), script, or any other entity comprising a set of instructions to be performed. When a source program, then the program may be translated via a compiler, assembler, interpreter, or the like, so as to operate properly in connection with the operating system. Furthermore, the software can be written as (a) an object oriented programming language, which has classes of data and methods, or (b) a procedure programming language, which has routines, subroutines, and/or functions, for example but not limited to, C, C++, Python, Java, among others. The software may be embodied in a computer program product, which may be a non-transitory computer readable medium or other medium.

The input interface 38 comprises an interface for entry of user input, such as a button or microphone or sensor (e.g., to detect user input). The input interface 38 may serve as a communications port for downloaded information to the wearable device 12 (such as via a wired connection). The output interfaces 40 comprises an interface for the presentation or transfer of data, such as a display or communications interface for the transfer (e.g., wired) of information stored in the memory, or to enable one or more feedback devices, such as lighting devices (e.g., LEDs), audio devices (e.g., tone generator and speaker), and/or tactile feedback devices (e.g., vibratory motor). In some embodiments, at least some of the functionality of the input and output interfaces 38 and 40 may be combined.

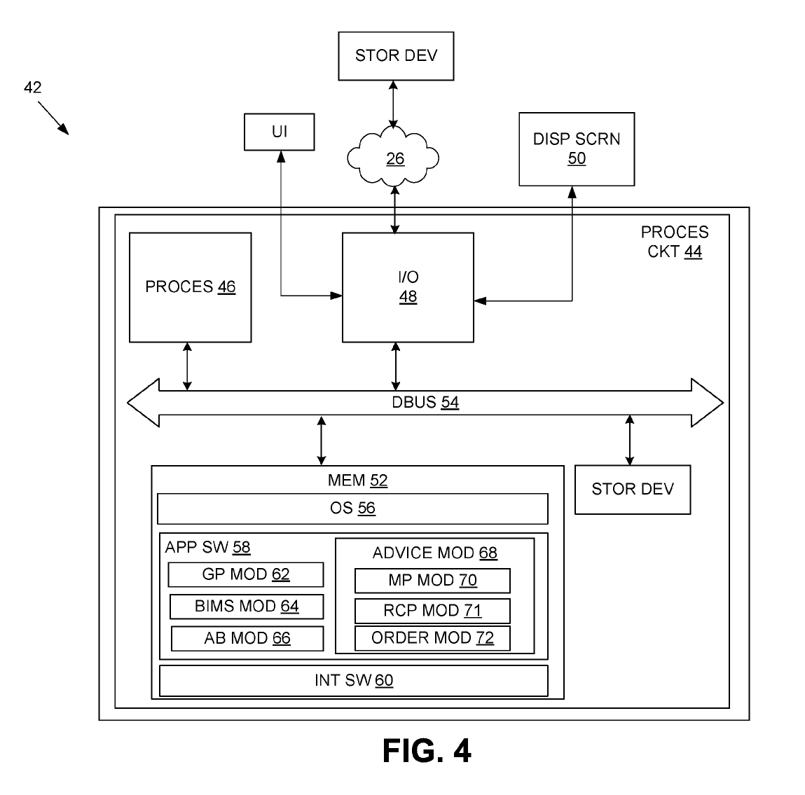

Having described the underlying hardware and software of the wearable device 12, attention is now directed to FIG. 4, which illustrates circuitry for an example computing device 42 of the computing system 28, in accordance with an embodiment of the invention. The computing device 42 may be embodied as an application server, computer, among other computing devices, and is also generally referred to herein as an apparatus. One having ordinary skill in the art should appreciate in the context of the present disclosure that the example computing device 42 is merely illustrative of one embodiment, and that some embodiments of computing devices may comprise fewer or additional components, and/or some of the functionality associated with the various components depicted in FIG. 4 may be combined, or further distributed among additional modules or computing devices, in some embodiments. Note that in some embodiments, one or more of the functionality of the computing device 42 may reside at the computing device 14, whether local to the child or parent or guardian or residing remotely (e.g., in a cloud computing or other remote computing environment). The computing device 42 is depicted in this example as a computer system, such as one providing a function of an application server. It should be appreciated that certain well-known components of computer systems are omitted here to avoid obfuscating relevant features of the computing device 42. In one embodiment, the computing device 42 comprises a processing circuit 44 (PROCES CKT) that comprises one or more processors, such as processor 46 (PROCES), input/output (I/O) interface(s) 48 (I/O), which in one embodiment is optionally coupled to a display screen 50 (DISP SCRN) and other user interfaces (e.g., keyboard, mouse, microphone, etc.), and memory 52 (MEM), all coupled to one or more data busses, such as data bus 54 (DBUS). In some embodiments, the display screen 50 (and/or user interface (UI)) may be coupled directly to the data bus 54. The memory 52 may include any one or a combination of volatile memory elements (e.g., random-access memory RAM, such as DRAM, and SRAM, etc.) and nonvolatile memory elements (e.g., ROM, Flash, solid state, EPROM, EEPROM, hard drive, tape, CDROM, etc.). The memory 52 may store a native operating system, one or more native applications, emulation systems, or emulated applications for any of a variety of operating systems and/or emulated hardware platforms, emulated operating systems, etc. In some embodiments, a separate storage device (STOR DEV) may be coupled to the data bus 54 or as a network-connected device (or devices) via the I/O interfaces 48 and the Internet 26. The storage device may be embodied as persistent memory (e.g., optical, magnetic, and/or semiconductor memory and associated drives) to store child data (e.g., based on questionnaires, recorded data communicated from the wearable device 12, and/or via data entered in web pages accessed at the electronics devices 18, 20, 22).

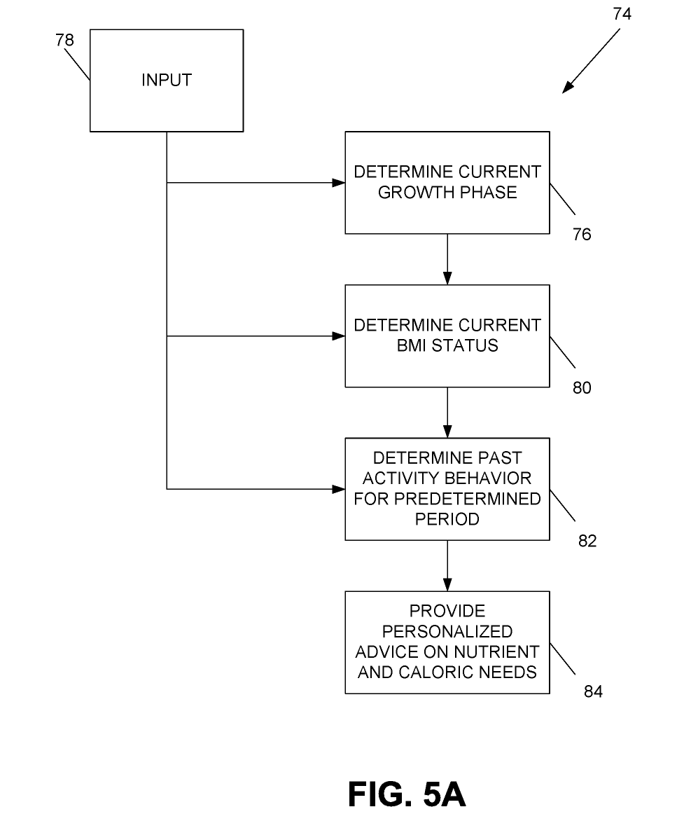

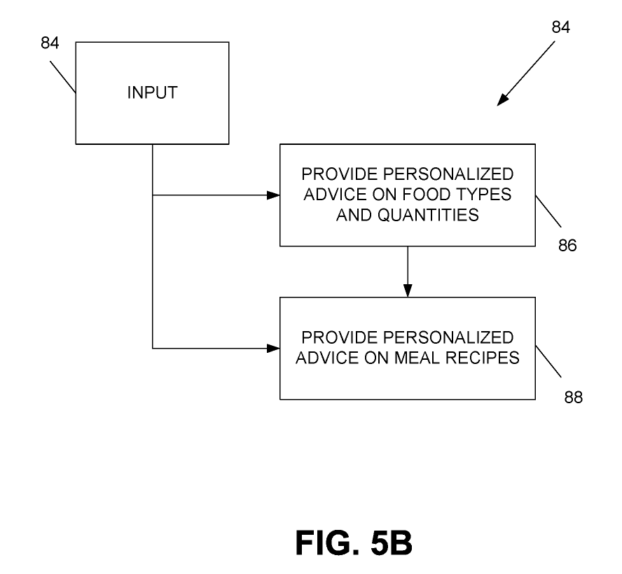

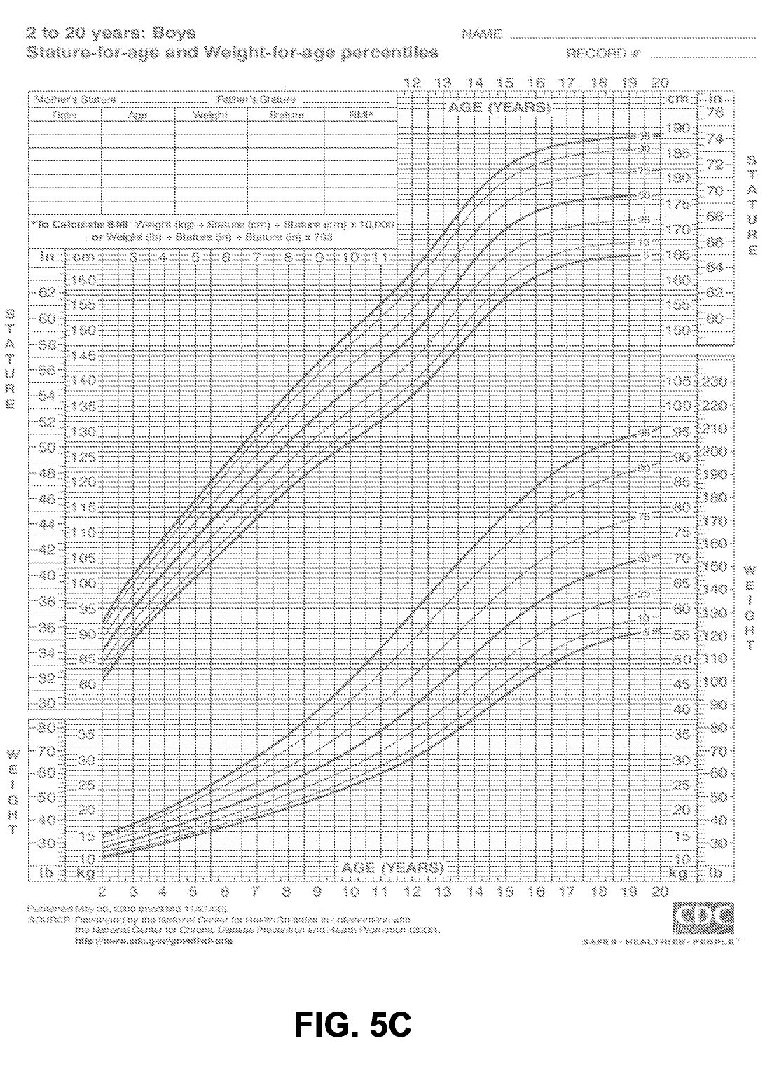

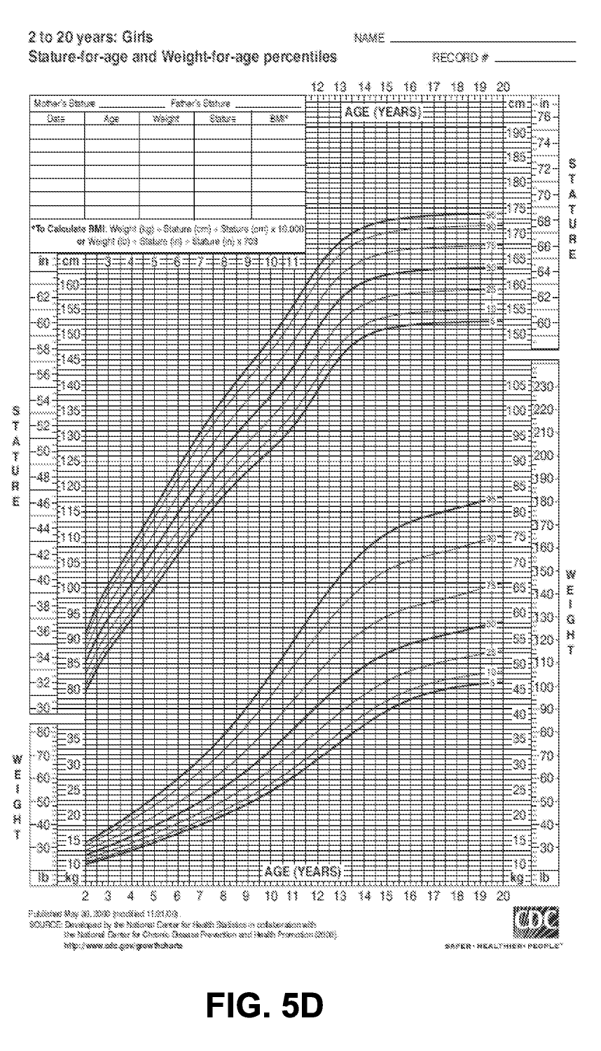

In the embodiment depicted in FIG. 4, the memory 52 comprises an operating system 56 (OS), application software 58 (APP SW), and interface software 60 (INT SW), the latter for enabling communications among network-connected devices and providing web and/or cloud services, among other software such as one or more APIs. The application software 58 comprises executable code in the form of a growth phase (GP) module 62 (GP MOD), a body mass index (BMI) status module 64 (BMIS MOD) an activity behavior (AB) module (AB MOD) 66, and an advice module 68 (ADV MOD), which in one embodiment comprises a meal planning (MP) module 70 (MP MOD), a recipe module 71 (RCP MOD), and an ordering module 72 (ORD MOD). With continued reference to FIG. 4, attention is also directed to FIGS. 5A-5B, which illustrate an example process 74 by which the nutrition coaching system receives and provides personalized advice on nutrient and caloric needs. Stated otherwise, the example process 74 corresponds to the underlying functionality of the software modules 62-70 of the application software 58. The GP module 62 corresponds to sub-process 76, which determines a current growth phase of the child. The GP module 62 receives inputs 78 and determines from the inputs 78 which phase among a plurality of phases (e.g., baby, toddler, preschooler, school age, teenager) the child is in. Note that there may be additional categories of growth phases in some embodiments, and in some embodiments, two or more of the phases may be combined. The inputs 78 include growth data, such as age and a dimension such as weight, height, girth, among other inputs 78 (e.g., gender, race, etc.). These inputs may be received manually (e.g., parent or child input at a computing device) and/or via communications entered over a network 26 and received at the I/O interfaces 48. As described earlier, the growth data may be received via a growth tracking device 22 (FIG. 2), such as a weighing device or height meter or a combination thereof in a single device. Using dimensions, such as height, in addition to the age of the child, enables a more accurate determination of the growth phase compared to merely using age differentiation, since child growth curves per child can be very different. For instance, when the computing device 42 is local to the child (e.g., in the child's home), input 78 may be entered at a computer terminal or via a phone or other electronics device, or received over a wired or wireless medium from the wearable device 12 (FIG. 1) or via the Internet 26 from a medical facility or records data facility. For instance, entry may be via a web screen that is provided from a computing device of the remote computing system 28 (FIG. 1), or when run locally, via a graphics user interface (GUI) generated by the device. When the computing device 42 is located remotely from the parent/child, such as part of the remote computing system 28, the input may be received via devices coupled directly or indirectly to the Internet 26 and via the I/O interfaces 48, such as from the electronics devices 18, 20, 22 or from storage devices or other computing devices coupled to the Internet 26. Note that the GP module 62 may further vet the determination of the growth phase for the child based on comparisons of growth data for peer age groups.

The BMI status module 64 determines the current status corresponding to a current body mass index for the child based on the inputted growth data 78 according to the sub-process 80. For instance, the BMI value for the child may be obtained by the wearable device 12 (FIG. 1) and communicated as growth data to the BMI status module 64, or the BMI value may be determined by the BMI module based on growth data (e.g., weight and height) obtained by the wearable device 12 and/or electronic devices 18, 20, 22 and communicated to the BMI status module 64, or determined at the electronics devices 18, 20 and communicated to the BMI status module 64. In some embodiments, the BMI status module 64 may receive growth data corresponding to the BMI of the child from other sources, such as manual input (e.g., after a doctor visit) or accessed from a network storage device, such as one managed by a medical facility that provided care to the child or a records facility, where the data may be stored locally in a storage device of the computing device 42. The BMI status module 64 determines the status of the child, such as whether the child is obese, overweight, at normal weight, or underweight. In some embodiments, the determination of the status may be done externally to the computing device 42.

The AB module 66 determines the activity behavior of the child based on the inputs 78, according to the sub-process 82. For instance, the activity behavior corresponds to data (e.g., recorded physical activity) received by the wearable device 12 (FIG. 1), and communicated to the AB module 66. Based on the inputs 78, the AB module 66 determines an activity level among a plurality of activity levels, such as whether the child engages in sedentary behavior for a predetermined period of time (e.g., over the last few days, or over a week, or other periods), whether the child engages in normal activity, or very active activity. Other categories and/or additional categories or fewer categories of physical activity may be used, such as based on data corresponding to perspiration, VO2 max, heart rate of the child, etc. Note that the sub-processes 76, 80, and 82 may be done in a different order than shown in FIG. 5A, including in reverse order, or in some embodiments, concurrently or substantially concurrently. Further, the inputs 78 may be received by the processing circuit 44 at different times, regardless of when the advice computations are determined. For instance, growth data may be received less frequently than the activity behavior data. As a non-limiting set of examples, the growth data may be received as inputs 78 on a monthly or even quarterly basis, whereas the activity behavior data may be received daily. Variations in the frequency of receipt of the inputs 78 are contemplated to be within the scope of the disclosure.

Based on the determinations in the sub-processes 76-82, the advice module 68 provides personalized advice on nutrient and caloric needs according to the sub-process 84. For instance, the determinations by the GP module 62 (e.g., of the growth phase) in sub-process 76 are used by the advice module 68 to tailor or personalize the required nutrient ratio for the child to the growth phase. The determinations by the BMI status module 64 in the sub-process 80 are used by the advice module 68 to tailor or personalize nutrient and caloric needs to weight goals (e.g., to lose weight to reach a normal weight, etc.). The determinations by the AB module 66 according to sub-process 82 are used by the advice module 68 to tailor or personalize caloric needs to historical (e.g., past child activity behavior) activity levels. The advice module 68 may use the various charts, such as those shown in FIGS. 5C-5D (developed by the National Center for Health Statistics in collaboration with the National Center for Chronic Disease Prevention and Health Promotion (2000), found at http://www.cdc.gov/growthcharts, the boys and girls charts incorporated herein by reference in their entirety), among other charts such as those published by the USDA (USDA 2010 Guidelines for Americans and in particular, Appendices 5 and 6), or other government or private medical/health/research institutions, to provide the appropriate nutrient and/or caloric needs. In one embodiment, growth data and nutritional data and corresponding nutrient components for various categories of age, gender, height, weight, physical activity, BMI, etc. may be accessed by the computing device 42 and stored locally, or accessed as needed over the Internet 26 or other networks, with personalization applied based on the recorded activity behavior and growth data for the child. The output of the advice module 68 may be presented on the display screen 50 (e.g., if local to the child or parent), or as a web page presented on a user interface of one of the electronics devices 18, 20, and may include such information in textual, graphical, video, and/or audio format that conveys caloric requirement advice and nutrient requirement advice personalized for the child. Note that the nutritional requirements comprise plural nutrient components for one of a respective plurality of age groups, wherein a ratio for each of the plural nutrient components differs among the plurality of age groups. The advice module 68 provides updates to the advice based on short term changes in caloric needs (e.g., due to levels of activity, growth spurts, etc.) as well as long term changes in nutrient needs. For instance, in growth phases of zero to five years, children typically need high calories and nutrients (e.g., brain development requires considerable fat intake, albeit in small portions). From five to fifteen years, the requirements change, where low fat, high fiber diets are more appropriate, with low sugar and salt (e.g., children may consume less salt than adults). At fifteen years, the child typically needs to eat more than the adult to support growth, yet with a slow down in food intake once the growth spurt has passed.

Referring to FIG. 5B, the sub-process 84 of the advice module 68 may further be broken down by the functionality of the meal planning module 70 and the ordering module 72. The meal planning module 70 provides personalized advice on food types and quantities consistent with the nutritional and caloric intake requirements that are personalized for the child, according to sub-process 86. Inputs 90 may be received by the meal planning module 70, such as parent or child input corresponding to personal preferences, tastes, and/or allergies or other dietary constraints, and may be received before, during, or after receiving the inputs 78 (FIG. 5A). The input 90 may be received from the wearable device 12, or other devices or resources. The recipe module 71 operates according to sub-process 88, and provides for personalized advice on meal recipes, and likewise is based on personal preferences, tastes, allergies, etc. The meal planning module 70 and recipe module 71 may compare the nutritional and caloric intake requirements of the child with a data structure comprising information about nutrient components for various food (including ingredient) types (e.g., the information stored in a local storage device or a remote storage device or devices), and generate a visual representation of a plurality of meals and/or meal recipes per day, week, month, etc. that comply with the nutritional and caloric intake requirements for the child. Such meal plans/recipes may be updated as the growth data and activity behavior data changes (e.g., as the child develops or experiences a change in lifestyle).

Referring back to FIG. 5A, in one embodiment, the ordering module 72 is optional, and may be configured to generate a grocery list of ingredients and/or foods that are used to satisfy the meal plans or recipes. The ordering module 72 may automatically place an order with a local grocer or food delivery facility (e.g., via a network communication), or generate the list and prompt the parent (or child) for permission to execute the order, or in some embodiments, generate the list and make a suggestion to the parent or child to place an order. In some embodiments, the list may be generated, and the parent may choose to merely go shopping for the ingredients/food items for the meals/recipes.

Note that one or more of the functionality of the application software 58 may be entirely implemented at the computing device 42, or distributed among plural devices in some embodiments. Also, though delineated with separate modules 62-72, in some embodiments, functionality of two or more of the modules 62-72 may be combined in some embodiments.

Execution of the application software 58 (and associated modules 62-72) and interface software 60 may be implemented by the processor 46 under the management and/or control of the operating system 56. The processor 46 may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the computing device 42.

The I/O interfaces 48 comprise hardware and/or software to provide one or more interfaces to the Internet 26, as well as to other devices such as the display screen 50 and user interfaces. In other words, the I/O interfaces 48 may comprise any number of interfaces for the input and output of signals (e.g., analog or digital data) for conveyance of information (e.g., data) over various networks and according to various protocols and/or standards. The user interfaces may include a keyboard, mouse, microphone, speakers, immersive head set, etc., which enable input and/or output by an administrator or other user (e.g., parent, child, or other care giver).

When certain embodiments of the computing device 42 are implemented at least in part with software (including firmware), as depicted in FIG. 4, it should be noted that the software (e.g., such as the application software 58 and interface software 60) can be stored on a variety of non-transitory computer-readable medium for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the computing device 42 are implemented at least in part with hardware, such functionality may be implemented with any or a combination of the following technologies, which are all well-known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), relays, contactors, etc.

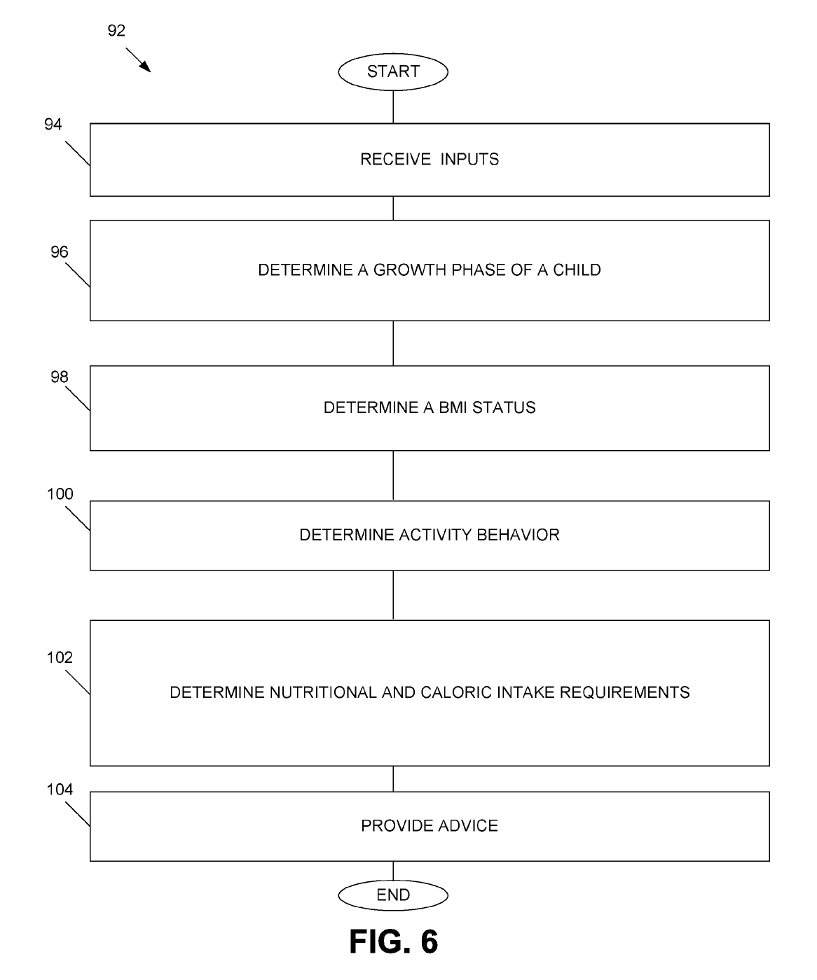

In view of the description above, it should be appreciated that one embodiment of a nutrition coaching method, depicted in FIG. 6 and referred to as a method 92 and encompassed between start and end designations, comprises receiving plural inputs corresponding to growth data, activity behavior data, and nutritional data (94); determining a growth phase of a child from among a plurality of growth phases based on the growth data of the child, the growth data comprising at least a current age and current dimension of the child (96); determining a status corresponding to a current body mass index for the child based on the growth data (98); determining activity behavior for the child (100); determining nutritional requirements and caloric intake requirements personalized for the child based on the determinations of the growth phase, the parameter, the activity behavior, and the nutritional data, the nutritional requirements comprising plural nutrient components for one of a respective plurality of age groups, wherein a ratio for each of the plural nutrient components differs among the plurality of age groups (102); and providing advice on the nutritional requirements and the caloric intake requirement personalized for the child (104).

Any process descriptions or blocks in the flow diagram of FIG. 6 should be understood as representing modules, segments, or portions of code which include one or more executable instructions for implementing specific logical functions or steps in the process, and alternate implementations are included within the scope of an embodiment of the present invention in which functions may be executed substantially concurrently, in a different order than depicted in FIG. 6, and/or additional logical functions or steps may be added, depending on the functionality involved, as would be understood by those reasonably skilled in the art of the present invention.

It should be noted that reference to a parent of the child is intended for brevity, and that a guardian, sibling, relative, friend, or other care giver may act on behalf of the child (alone or with the child) when inputting data manually, or when an output of data is presented.

In one embodiment, a claim to an apparatus is disclosed, the apparatus comprising a processing circuit configured to: receive plural inputs corresponding to growth data, activity behavior data, and nutritional data; determine a growth phase of a child from among a plurality of growth phases based on the growth data of the child, the growth data comprising at least a current age and current dimension of the child; determine a status corresponding to a current body mass index for the child based on the growth data; determine activity behavior for the child; determine nutritional requirements and caloric intake requirements personalized for the child based on the determinations of the growth phase, the status, the activity behavior, and nutritional data, the nutritional requirements comprising plural nutrient components for one of a respective plurality of age groups, wherein a ratio for each of the plural nutrient components differs among the plurality of age groups; and provide advice on the nutritional requirements and the caloric intake requirement personalized for the child.

The apparatus of the prior claim, wherein the processing circuit is configured to determine the activity behavior based on receiving the activity behavior data corresponding to a recorded physical activity level of the child defined according to one of a plurality of levels of physical activity, and wherein the processing circuit determines the status by determining whether the child is obese, overweight, normal weight, or underweight based on receiving the body mass index or based on deriving the body mass index from the growth data.

The apparatus of any one of the preceding claims, wherein the processing circuit is configured to provide meal planning recommendations personalized for the child based on the nutritional requirements and the caloric intake requirements, the meal planning recommendations comprising one or any combination of the following: food selection, food preparation, meal timing, food ingredients, food portions, relative food proportion, nutrient levels, and proportion of nutrients.

The apparatus of the prior claim, wherein the processing circuit is further configured to provide the meal planning recommendations based on additional input, wherein the meal planning recommendations for the child in a first growth phase of the plurality of growth phases are different than the meal planning recommendations for the child in a second growth phase of the plurality of growth phases.

The apparatus of any one of the preceding claims, wherein the processing circuit is further configured to determine the nutritional requirements and caloric intake requirements based on computing and comparing growth rates of the child over plural periods of time.

The apparatus of any one of the preceding claims, wherein the processing circuit is further configured to determine the growth phase by comparing growth data for peer age groups with the growth data of the child over the plurality of growth phases.

The apparatus of any one of the preceding claims, wherein the growth data includes one or any combination of weight, height, body mass index, gender, age, and girth of the child.

The apparatus of any one of the preceding claims, wherein the processing circuit is configured to receive the growth data based on manual input, sensor data, or a combination of manual input and sensor data.

The apparatus of any one of the preceding claims, wherein the processing circuit is configured to receive activity behavior data based on manual input, sensor data, or a combination of manual input and sensor data, wherein the processing circuit determines that the activity behavior falls within one of plural predefined categories of activity levels based on the activity behavior data.

The apparatus of any one of the preceding claims, wherein the processing circuit is coupled to a storage device that stores the nutritional data, the growth data, and the activity behavior data.

The apparatus of any one of the preceding claims, wherein the processing circuit is further configured to receive the growth data, behavioral data, and nutritional data over either the Internet, or over a wired or wireless connection from a co-located device.

The apparatus of any one of the preceding claims, wherein the processing circuit is further configured to cause an automated ordering of food corresponding to the meal planning recommendations.

In one embodiment, a method is disclosed, the method comprising: receiving plural inputs corresponding to growth data, activity behavior data, and nutritional data; determining a growth phase of a child from among a plurality of growth phases based on the growth data of the child, the growth data comprising at least a current age and current dimension of the child; determining a status corresponding to a current body mass index for the child based on the growth data; determining activity behavior for the child; determining nutritional requirements and caloric intake requirements personalized for the child based on the determinations of the growth phase, the parameter, the activity behavior, and the nutritional data, the nutritional requirements comprising plural nutrient components for one of a respective plurality of age groups, wherein a ratio for each of the plural nutrient components differs among the plurality of age groups; and providing advice on the nutritional requirements and the caloric intake requirement personalized for the child.

The method of the preceding claim, further comprising providing meal planning recommendations personalized for the child based on the nutritional requirements and the caloric intake requirements.

In one embodiment, disclosed is a computer program product that enables a processing circuit to carry out the aforementioned method.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms. Any reference signs in the claims should be not construed as limiting the scope.

1. An apparatus, comprising:
a processing circuit configured to:
receive plural inputs corresponding to growth data, activity behavior data, and nutritional data;
determine a growth phase of a child from among a plurality of growth phases based on the growth data of the child, the growth data comprising at least a current age and current dimension of the child;
determine a status corresponding to a current body mass index for the child based on the growth data;
determine activity behavior for the child;
determine nutritional requirements and caloric intake requirements personalized for the child based on the determinations of the growth phase, the status, the activity behavior, and nutritional data, the nutritional requirements comprising plural nutrient components for one of a respective plurality of age groups, wherein a ratio for each of the plural nutrient components differs among the plurality of age groups; and
provide advice on the nutritional requirements and the caloric intake requirement personalized for the child.

2. The apparatus of claim 1, wherein the processing circuit is configured to determine the activity behavior based on receiving the activity behavior data corresponding to a recorded physical activity level of the child defined according to one of a plurality of levels of physical activity, and wherein the processing circuit determines the status by determining whether the child is obese, overweight, normal weight, or underweight based on receiving the body mass index or based on deriving the body mass index from the growth data.

3. The apparatus of claim 1, wherein the processing circuit is configured to provide meal planning recommendations personalized for the child based on the nutritional requirements and the caloric intake requirements, the meal planning recommendations comprising one or any combination of the following: food selection, food preparation, meal timing, food ingredients, food portions, relative food proportion, nutrient levels, and proportion of nutrients.

4. The apparatus of claim 3, wherein the processing circuit is further configured to provide the meal planning recommendations based on additional input, wherein the meal planning recommendations for the child in a first growth phase of the plurality of growth phases are different than the meal planning recommendations for the child in a second growth phase of the plurality of growth phases.

5. The apparatus of claim 1, wherein the processing circuit is further configured to determine the nutritional requirements and caloric intake requirements based on computing and comparing growth rates of the child over plural periods of time.

6. The apparatus of claim 1, wherein the processing circuit is further configured to determine the growth phase by comparing growth data for peer age groups with the growth data of the child over the plurality of growth phases.

7. The apparatus of claim 1, wherein the growth data includes one or any combination of weight, height, body mass index, gender, age, and girth of the child.

8. The apparatus of claim 1, wherein the processing circuit is configured to receive the growth data based on manual input, sensor data, or a combination of manual input and sensor data.

9. The apparatus of claim 1, wherein the processing circuit is configured to receive activity behavior data based on manual input, sensor data, or a combination of manual input and sensor data, wherein the processing circuit determines that the activity behavior falls within one of plural predefined categories of activity levels based on the activity behavior data.

10. The apparatus of claim 1, wherein the processing circuit is coupled to a storage device (STOR DEV) that stores the nutritional data, the growth data, and the activity behavior data.

11. The apparatus of claim 1, wherein the processing circuit is further configured to receive the growth data, behavioral data, and nutritional data over either the Internet, or over a wired or wireless connection from a co-located device.

12. The apparatus of claim 1, wherein the processing circuit is further configured to cause an automated ordering of food corresponding to the meal planning recommendations.

13. A method, comprising:
receiving, at a processor, plural inputs corresponding to growth data, activity behavior data, and nutritional data;
determining, by the processor, a growth phase of a child from among a plurality of growth phases based on the growth data of the child, the growth data comprising at least a current age and current dimension of the child;
determining, by the processor, a status corresponding to a current body mass index for the child based on the growth data;
determining, by the processor, activity behavior for the child;
determining, by the processor, nutritional requirements and caloric intake requirements personalized for the child based on the determinations of the growth phase, the parameter, the activity behavior, and the nutritional data, the nutritional requirements comprising plural nutrient components for one of a respective plurality of age groups, wherein a ratio for each of the plural nutrient components differs among the plurality of age groups; and
providing, via a display, advice on the nutritional requirements and the caloric intake requirement personalized for the child.

14. The method of claim 13, further comprising providing, via a display, meal planning recommendations personalized for the child based on the nutritional requirements and the caloric intake requirements.

15. A computer program product that enables a processing circuit to carry out the method of claim 14.
